(19) United States
(12) Patent Application Publication
(10) Pub. No.: US 2021/0009935 A1
(43) Pub. Date: Jan. 14, 2021

(54) CELL CULTURE MONITORING DEVICE AND CELL CULTURE SYSTEM

(71) Applicant: Hitachi, Ltd., Tokyo (JP)

(72) Inventors: Hikaru SAITO, Tokyo (JP); Kakuro HIRAI, Tokyo (JP); Midori KATO, Tokyo (JP); Masaharu KIYAMA, Tokyo (JP); Kunio OHYAMA, Tokyo (JP); Hiroko HANZAWA, Tokyo (JP); Shizu TAKEDA, Tokyo (JP)

(73) Assignee: HITACHI, LTD.

(21) Appl. No.: 16/924,955

(22) Filed: Jul. 9, 2020

(30) Foreign Application Priority Data
Jul. 12, 2019 (JP) 2019-130406

(51) Int. Cl. C12M 1/34; G01N 33/543; C12M 1/36

(52) U.S. Cl. CPC C12M 41/36; C12M 41/48; G01N 33/54366

(57) ABSTRACT

An object of the invention is to provide a cell culture monitoring device capable of monitoring the number of cells during culture and a cell culture system. The cell culture monitoring device for monitoring proliferation of cells includes: a detection unit configured to detect particles of exosomes in culture supernatant; and an analysis unit configured to calculate the number of cells based on an obtained detection result. The cell culture system includes the cell culture monitoring device and an automatic culture device.

### BACKGROUND OF THE INVENTION

### 1. Field of the Invention

The present invention relates to a cell culture monitoring device and a cell culture system.

### 2. Description of the Related Art

Regenerative medicine is new medicine that can artificially create cells and tissues and transplant them into a body to completely restore functions of defective cells and tissues, and can cure disorders and diseases that cannot be treated by medicine of the related art. Cells expected as source cells in the regenerative medicine are pluripotent stem cells such as ES cells and iPS cells. Since the pluripotent stem cells proliferate indefinitely and have pluripotency that can differentiate into almost all types of cells, the pluripotent stem cells have a high potential for industrial application.

Cell culture used for the regenerative medicine is performed in a cell culture clean room called a cell processing center (CPC) according to a good manufacturing practice (GMP). Here, since the cell culture is performed manually by an engineer, cell preparation is very labor intensive and costly. Further, since the cell culture is performed manually, there is a risk of biological contamination. As means for solving these problems, a device for automating a cell culture process in a closed system has been developed. In an automatic culture device, by using a closed culture container that does not require operations of opening and closing a lid of the culture container, automation of the cell culture process and reduction of the risk of the biological contamination can be achieved.

In the cell culture process, cells proliferate by repeating division, but when culture environment deteriorates, the number of cells causing cell death increases, and if the state continues, all cells may die. Further, the stem cells such as iPS cells need to proliferate while maintaining an undifferentiated state, but under conditions where cells are stressed, the stem cells easily deviate from the undifferentiated state and lose a differentiation ability that is a function of the stem cells. Further, in a case of inducing differentiation of the stem cells such as iPS cells into target cells, if the induction of the differentiation of the stem cells into the target cells does not proceed efficiently, a yield of a final product will decrease. Therefore, in order to stabilize production and improve quality using the automatic culture device of cells, it is important to optimize a culture state by monitoring the culture state and adding control according to the culture state. In particular, in a case of a closed type automatic culture device, it is necessary to evaluate the culture state while maintaining a closed space in order to maintain sterility. Examples of a culture state evaluation method in a closed system include cell observation or image analysis using an optical camera image obtained by a phase contrast microscope or the like (for example, JP-A-2011-229409 and JP-A-2018-57401).

In addition, a method of monitoring culture by measuring components in a culture solution is also known. The method is a technique for aseptically extracting culture supernatant during the culture and measuring components to be measured using a high-performance liquid chromatograph or the like, and the method is performed when cells are used to produce pharmaceutical such as antibody pharmaceutical (for example, JP-A-2010-187594).

In order to monitor the cell culture state while maintaining the closed space to maintain the sterility, it is necessary to evaluate the culture state in-line without taking out the culture supernatant or cells from the closed space to the outside.

On the other hand, in a case of adherent cells or cells that form cell clusters, the number of cultured cells is basically obtained by detaching cells using an enzyme treatment with trypsin or the like and counting the number of cells, and in this case, it is difficult to monitor the number of cells during the culture because the number of cells cannot be automatically measured.

### SUMMARY OF THE INVENTION

An object of the invention is to provide a cell culture monitoring device capable of monitoring the number of cells during culture and a cell culture system.

According to an embodiment of the invention, a cell culture monitoring device for monitoring proliferation of cells includes: a detection unit configured to detect particles of exosomes in culture supernatant; and an analysis unit configured to calculate the number of cells based on an obtained detection result. In the detection unit, an amount of antibodies that bind to markers of the exosomes may be measured, and in the analysis unit, the number of cells may be calculated based on the amount of antibodies. In the detection unit, an amount of antibodies that bind to markers of the exosomes may be measured, and in the analysis unit, particle density of the exosomes may be calculated based on the amount of antibodies, and the number of cells may be calculated based on the particle density. In the detection unit, the number of particles of the exosomes may be measured, and in the analysis unit, the number of cells may be calculated based on the number of particles.

According to another embodiment of the invention, a cell culture system includes: the cell culture monitoring device according to any one of the above; and an automatic culture device. Data of the number of cells calculated by the analysis unit may be transmitted to the automatic culture device, for example, within 1 hour after calculation.

According to a further embodiment of the invention, a method for measuring the number of cells in cultured cells includes: a detection step of detecting exosomes in culture supernatant; and a calculation step of calculating the number of cells based on a detection result. In the detection step, an amount of antibodies that bind to markers of the exosomes maybe measured, and in the calculation step, the number of cells may be calculated based on the amount of antibodies. In the detection step, an amount of antibodies that bind to markers of the exosomes may be measured, and in the calculation step, particle density of the exosomes may be calculated based on the amount of antibodies, and the number of cells may be calculated based on the particle density. In the detection step, the number of particles of the exosomes may be measured, and in the calculation step, the number of cells may be calculated based on the number of particles.

According to a further embodiment of the invention, a program for causing a cell culture monitoring device to perform the method for measuring the number of cells is provided.

According to a further embodiment of the invention, a computer-readable storage medium storing the program is provided.

According to the invention, a cell culture monitoring device capable of monitoring the number of cells during culture and a cell culture system can be provided.

### BRIEF DESCRIPTION OF THE DRAWINGS

FIG. 1 is a schematic diagram showing a configuration of a cell culture monitoring device according to an embodiment of the invention.

FIG. 2 is a schematic diagram showing a configuration of a cell culture system according to the embodiment of the invention.

FIG. 3 is a flowchart of a method for controlling the cell culture system according to the embodiment of the invention.

FIG. 4 is a graph showing changes in the number of exosome granules per amount of culture supernatant in a proliferation culture process of iPS cells according to an example of the invention.

FIG. 5 is a graph showing a correlation between the number of exosome granules and the number of cells per amount of culture supernatant in the proliferation culture process of the iPS cells according to the example of the invention.

### DESCRIPTION OF THE PREFERRED EMBODIMENTS

Various embodiments of the invention will be described below with reference to the drawings and examples. However, these embodiments are only for implementing the invention, and do not limit the technical scope of the invention. In the drawings, common components are denoted by the same reference numerals.

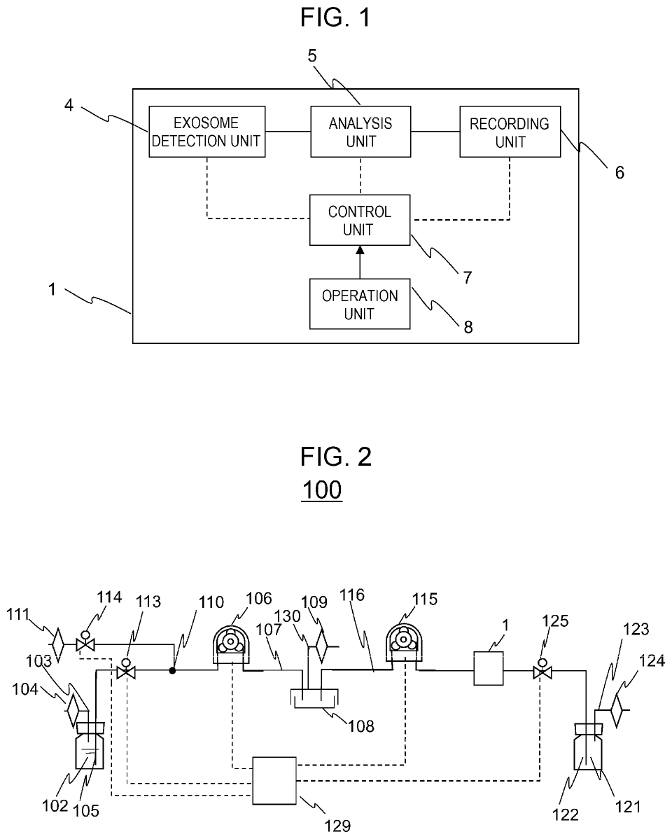

==Cell Culture Monitoring Device==A cell culture monitoring device according to the invention can measure, analyze, and record exosomes as shown in FIG. 1. A cell culture monitoring device 1 shown in FIG. 1 includes a detection unit 4 for detecting exosomes, an analysis unit 5 for analyzing a detection result, a recording unit 6 for recording analyzed data or the like, a control unit 7 for controlling the detection unit 4, the analysis unit 5, and the recording unit 6, and an operation unit 8 capable of operating the control unit 7. Although the device includes only one detection unit in FIG. 1, the device may include a plurality of exosome detection units. In this case, a plurality of samples can be measured simultaneously. Cells used for monitoring are not particularly limited, and examples of the cells include pluripotent stem cells such as iPS cells and ES cells, stem cells such as mesenchymal stem cells, and other human-derived cells and animal-derived cells, and the cells may be cultured cells or primary cultured cells.

### ==Cell Culture System==

As shown in FIG. 2, the cell culture monitoring device 1 may be connected to a closed type automatic culture device 100 to constitute a cell culture system as a whole . The closed type automatic culture device is not particularly limited, a device already developed may be applied, and an example is shown below.

The cell culture system of the present disclosure includes a first container 102 for containing a first liquid and a second container 108 for containing the first liquid. The first container 102 is a container for storing a medium for cell culture, which is the first liquid. The second container 108 is a container for cell culture, and is not particularly limited in a shape such as a dish or a bottle. The first container 102 can be easily manufactured according to the technical common sense of those skilled in the art in consideration of the purpose. The first container 102 includes an air pressure control tube 103 that is open to the outside air, and has an end in a gas phase inside the container. The second container 108 can also be easily manufactured according to the technical common sense of those skilled in the art in consideration of the purpose. The second container 108 includes an air pressure control tube 130 that is open to the outside air, and has an end in a gas phase inside the container.

The closed type automatic culture device 100 includes a first liquid feed tube 105 for feeding the first liquid in the first container 102 and a second liquid feed tube 107 for feeding the first liquid in the first liquid feed tube 105 to the second container 108. The second liquid feed tube 107 includes a first liquid feed pump 106, and controls liquid feed in the second liquid feed tube 107. Each of the liquid feed tubes can be easily manufactured according to the technical common sense of those skilled in the art. The first liquid feed tube 105 includes a first valve 113 and a second valve 114, and can switch presence and absence of the liquid feed by opening and/or closing each of the first valve 113 and the second valve 114.

Further, the closed type automatic culture device 100 includes a third container 121 for discarding the first liquid in the second container 108. The third container 121 can be easily manufactured according to the technical common sense of those skilled in the art in consideration of the purpose. The third container 121 includes an air pressure control tube 123 that is open to the outside air, and has an end in a gas phase inside the container.

The closed type automatic culture device 100 further includes a third liquid feed tube 116 for discharging the first liquid in the second container 108 and a fourth liquid feed tube 122 that is connected to the third liquid feed tube 116 and discharges the first liquid in the second container 108 to the third container 121 through the third liquid feed tube 116. The third liquid feed tube 116 includes a second liquid feed pump 115, and controls liquid feed in the third liquid feed tube 116. Each of the liquid feed tubes can be easily manufactured according to the technical common sense of those skilled in the art. The fourth liquid feed tube 122 includes a third valve 125, and by opening and/or closing the third valve 125, can switch presence and absence of the liquid feed and can start and stop measurement of the cell culture monitoring device 1.

The closed type automatic culture device 100 may independently have a control unit 129, and it is preferable that the closed type automatic culture device 100 can automatically control an operation of a pump and opening and/or closing of a valve.

### ==Method for Measuring the Number of Cells==

A method for measuring the number of cultured cells of the present disclosure includes a detection step of detecting exosomes in culture supernatant and a calculation step of calculating the number of cells based on a detection result.

Here, a method for detecting the exosomes is not particularly limited, and a method using markers of exosomes, an electric resistance nanopulse method, a nanoparticle tracking method, a dynamic light scattering method, and a method using infrared spectroscopy or Raman spectroscopy can be exemplified. The detection result obtained in the detection step maybe one that correlates with both the number of exosome particles and the number of cells, and an expression level of the markers and the number of exosome particles can be exemplified.

When the markers are used, the method may include, for example, a step of obtaining culture supernatant, a step of causing the obtained culture supernatant to contact with antibodies of the markers, and a step of detecting antibodies bound to the exosomes. These steps can be performed automatically. The markers used here are not particularly limited, and Alix, CD24, CD63, CD81, CD9, and TSG101 can be exemplified. A method for detecting the antibodies is not particularly limited, and an ELISA and an electrophoresis method can be used.

In the calculation step, particle density of the exosomes in the culture supernatant may be calculated based on the detection result of the exosomes such as the expression level of the markers, and the particle density can be calculated based on the expression level of the markers by creating a standard curve of the particle density and the expression level of the markers in advance. The expression level of the markers may be a measured value using the antibodies. In the electric resistance nanopulse method or the like, the number of particles can be obtained as the detection result, and thus the particle density can be easily obtained. Then, the number of cells can be calculated based on the particle density by creating a standard curve of the particle density and the number of cells in advance.

Further, in the calculation step, the number of cells can be directly calculated based on the expression level of the markers by creating a standard curve of the expression level of the markers and the number of cells in advance. Here, when the antibodies of the markers are used for detecting the markers, a detected value of an amount of bound antibodies may be used as the expression level of the markers.

### ==Operation Method of Cell Culture System==

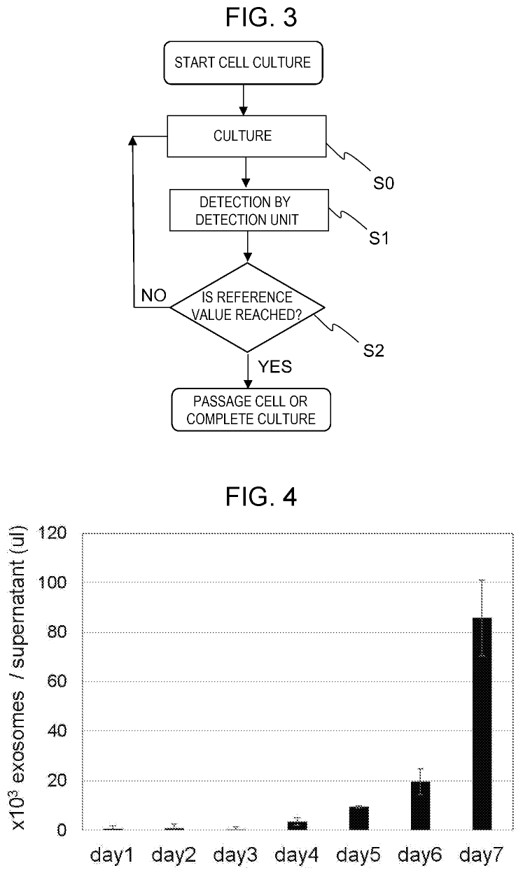

An operation method of the cell culture system will be described in detail below. Control of the cell culture system may be performed manually or by the control unit 7. FIG. 3 shows a flowchart when the control unit 7 is used to perform the control.

First, cell culture is started and the culture is continued (S0). After culturing for a predetermined period, for exosomes in the cell supernatant, the exosomes are detected by the detection unit 4 using the method described above (S1). For example, when using markers for the detection of the exosomes, by recording a standard curve of the particle density and a detected amount of antibodies against the markers in the recording unit, the analysis unit 5 can calculate the particle density of the exosomes based on a measured value of the detected amount of the antibodies. Then, by recording the standard curve of the particle density and the number of cells in the recording unit, the analysis unit 5 can calculate the number of cells based on the obtained particle density. Here, without calculating the particle density of the exosomes, the number of cells can be directly calculated based on the measured value of the detected amount of the antibodies by the analysis unit 5 as long as a standard curve of the detected amount of the antibodies and the number of cells is recorded in the recording unit. When the electrical resistance nanopulse method or the like is used for detecting the exosomes, the number of particles can be obtained, and thus the analysis unit 5 only needs to calculate the particle density. Then, by recording the standard curve of the particle density and the number of cells in the recording unit, the analysis unit 5 can calculate the number of cells based on the obtained particle density.

When the number of cells does not reach a predetermined reference value (S2), data of the number of cells calculated by the analysis unit of the cell culture monitoring device is transmitted to the automatic culture device, for example, within 12 hours, preferably within 6 hours, more preferably within 3 hours, and further preferably within 1 hour after the calculation, and the cell culture is continued. When the number of cells has reached the predetermined reference value (S2), the cells are passaged directly, or the data of the number of cells is transmitted to the automatic culture device, and the cell culture is completed.

A program for performing such a method and a computer-readable storage medium storing the program may also fall within the scope of the invention.

### EXAMPLE 1

In an example, it is shown that the number of exosome granules in the culture supernatant increases over time according to the cell culture process, and the number of exosome granules and the number of cells have a high correlation. Specifically, iPS cell line 201B7 was used to perform culture for 1 week, and the number of exosome granules and the number of cells were monitored. Conditions used for ordinary iPS cells were applied as cell culture conditions.

During a culture period of the iPS cells, for culture supernatant collected every day, an expression level of CD63, which is a marker of an exosome, was quantified by an ELISA method, and the number of exosome granules was calculated by using a standard curve, which is created in advance, of the expression level of CD63 and the number of exosome granules. FIG. 4 shows a relationship between the number of exosome granules per amount of culture supernatant and the number of culture days after cell seeding. As is clear from the graph, the number of exosome granules increases as the culture period in the culture supernatant becomes longer. Thus, as the cells proliferate, the number of exosome granules increases.

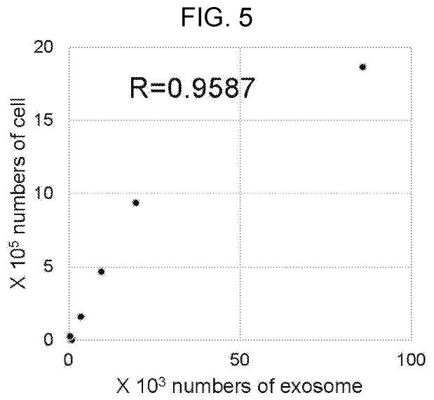

Next, for the number of cells simultaneously measured, a relationship between the number of cells and the number of exosome granules is shown in FIG. 5.

As is clear from the graph, there is a correlation between the number of cells and the number of exosome granules. Then, a correlation coefficient between the number of cells and the number of exosome granules was calculated to be 0.9587, which indicates a high correlation. Thus, the number of exosome granules can be an index of cell proliferation.

The invention is useful as a cell culture monitoring device when culturing cells.

What is claimed is:

1. A cell culture monitoring device for monitoring proliferation of cells, the cell culture monitoring device comprising:
a detection unit configured to detect particles of exosomes in culture supernatant; and
an analysis unit configured to calculate the number of cells based on an obtained detection result.

2. The cell culture monitoring device according to claim 1, wherein
in the detection unit, an amount of antibodies that bind to markers of the exosomes is measured, and
in the analysis unit, the number of cells is calculated based on the amount of antibodies.

3. The cell culture monitoring device according to claim 1, wherein
in the detection unit, an amount of antibodies that bind to markers of the exosomes is measured, and
in the analysis unit, particle density of the exosomes is calculated based on the amount of antibodies, and the number of cells is calculated based on the particle density.

4. The cell culture monitoring device according to claim 1, wherein
in the detection unit, the number of particles of the exosomes is measured, and
in the analysis unit, the number of cells is calculated based on the number of particles.

5. A cell culture system comprising:
the cell culture monitoring device according to claim 1; and
an automatic culture device.

6. A cell culture system comprising:
the cell culture monitoring device according to claim 2; and
an automatic culture device.

7. A cell culture system comprising:
the cell culture monitoring device according to claim 3; and
an automatic culture device.

8. A cell culture system comprising:
the cell culture monitoring device according to claim 4; and
an automatic culture device.

9. The cell culture system according to claim 5, wherein
data of the number of cells calculated by the analysis unit is transmitted to the automatic culture device.

10. The cell culture system according to claim 9, wherein
the data of the number of cells is transmitted to the automatic culture device within 1 hour after calculation.

11. A method for measuring the number of cells in cultured cells, the method comprising:
a detection step of detecting exosomes in culture supernatant; and
a calculation step of calculating the number of cells based on a detection result.

12. The method for measuring the number of cells according to claim 11, wherein
in the detection step, an amount of antibodies that bind to markers of the exosomes is measured, and
in the calculation step, the number of cells is calculated based on the amount of antibodies.

13. The method for measuring the number of cells according to claim 11, wherein
in the detection step, an amount of antibodies that bind to markers of the exosomes is measured, and
in the calculation step, particle density of the exosomes is calculated based on the amount of antibodies, and the number of cells is calculated based on the particle density.

14. The method for measuring the number of cells according to claim 11, wherein
in the detection step, the number of particles of the exosomes is measured, and
in the calculation step, the number of cells is calculated based on the number of particles.

15. A computer-readable non-transitory storage medium storing the program for causing a cell culture monitoring device to perform the method for measuring the number of cells according to claim 11.

16. A computer-readable non-transitory storage medium storing the program for causing a cell culture monitoring device to perform the method for measuring the number of cells according to claim 12.

17. A computer-readable non-transitory storage medium storing the program for causing a cell culture monitoring device to perform the method for measuring the number of cells according to claim 13.

18. A computer-readable non-transitory storage medium storing the program for causing a cell culture monitoring device to perform the method for measuring the number of cells according to claim 14.
